(19) United States
(12) Patent Application Publication
(10) Pub. No.: US 2026/0076833 A1
(43) Pub. Date: Mar. 19, 2026

(54) UV ABSORBER ALTERATION SYSTEM AND METHOD

(71) Applicant: Perfect IP, LLC, Dallas, TX (US)

(72) Inventors: Steven Edward SMATHERS, Dallas, TX (US); Ruth SAHLER, Irvine, CA (US); Sean ENRIGHT, Irvine, CA (US); Somaly CHHOEUNG, Irvine, CA (US)

(21) Appl. No.: 19/051,079

(22) Filed: Feb. 11, 2025

Related U.S. Application Data
(60) Provisional application No. 63/696,041, filed on Sep. 18, 2024.

(51) Int. Cl. A61F 9/008

(52) U.S. Cl. CPC A61F 9/00812

(57) ABSTRACT

A system for forming an intraocular lens (IOL). The system includes a pulsed laser configured to produce a pulsed laser output and an optical device configured to form a focused laser output from the pulsed laser output and direct the focused laser output at an IOL containing a UV absorber compound. The focused laser output is configured to alter molecular bonds of the UV absorber compound to form a new or modified compound within the IOL that imparts a different refractive property in the IOL.

### RELATED APPLICATIONS

The present disclosure claims priority to U.S. Provisional Patent App Ser. No. 63/696,041, filed Sep. 18, 2024, which is incorporated herein by reference in its entirety for all purposes.

### TECHNICAL FIELD

The present disclosure relates to intraocular lenses (IOLs). More particularly, and not by way of limitation, the present disclosure is directed to an apparatus, system or method for creating an intraocular lens with ultraviolet-absorbing chromophores, referred to herein as “UV absorbers” for the sake of simplicity, which can be altered to change the refractive characteristics of the IOL.

### BACKGROUND

IOLs are artificial lenses implantable into the eye for improving vision. In general, there are two types of IOLs, phakic lenses and pseudophakic lenses. Phakic lenses can be implanted in the anterior or posterior chamber of the eye and are meant to remediate the refractive errors of the eye's natural lens. Pseudophakic lenses are meant to replace the eye's natural lens and are typically used during cataract surgery.

IOLs can be constructed from a variety of materials including acrylic polymers, poly(methyl methacrylate) (PMMA), silicon, and hydrogels. Many IOLs include UV absorbers, which are added by the lens manufacturer to absorb UV light and protect the retina and other structures of the eye after implantation.

Two common techniques for forming IOLs include molding and machining. When molded, an optical polymeric material with a predetermined diopter power is formed in an injection mold with a desired shape. These IOLs lenses are available in standard diopter powers, typically differing by steps of about 0.5 diopter power. The machining process involves lathing and milling a blank into a desired shape at low temperatures, e.g., −10° F.

### SUMMARY

The present disclosure pertains to a system for forming a refractive lens. The system includes a pulsed laser configured to produce a pulsed laser output and an optical device configured to form a focused laser output from the pulsed laser output and direct the focused laser output to a polymeric lens material containing a UV absorber compound. The focused laser output is configured to alter molecular bonds of the UV absorber compound to form a new or modified compound within the IOL that imparts a different refractive property in the refractive lens. In some embodiments, the refractive lens is an IOL.

Other aspects, embodiments and features of the present disclosure will become apparent from the following detailed description when considered in conjunction with the accompanying figures. In the figures, each identical, or substantially similar component that is illustrated in various figures is represented by a single numeral or notation. For purposes of clarity, not every component is labeled in every figure. Nor is every component of the disclosure shown where illustration is not necessary to allow those of ordinary skill in the art to understand the disclosure.

### BRIEF DESCRIPTION OF THE DRAWINGS

The novel features believed characteristic of the disclosure are set forth in the appended claims. The disclosure itself, however, as well as a preferred mode of use, further objectives and advantages thereof, will be best understood by reference to the following detailed description of illustrative embodiments when read in conjunction with the accompanying drawings, wherein:

FIG. 1 is a perspective view of an intraocular lens (IOL);

FIG. 2 is a side view of an IOL;

FIG. 3 is a sectioned perspective view of a lens formed from the application of laser radiation according to an illustrative embodiment;

FIG. 4 is a side view of the lens depicted in FIG. 3;

FIG. 5 is a block diagram of a system for creating an IOL according to an illustrative embodiment;

FIG. 6 is a schematic block diagram of the system for creating an IOL as a part of an overall system according to an illustrative embodiment;

FIGS. 7A and 8B illustrate a convex and biconvex lens that can be formed according to the present disclosure;

FIGS. 8A and 8B illustrate a concave and a biconcave lens that can be formed according to the present disclosure;

FIGS. 9A and 9B illustrate various phase wrapping lens structures that can be formed according to the present disclosure;

FIGS. 10A and 10B illustrate a plan view and a side view of refractive index patterns that can be formed according to the present disclosure;

FIGS. 11A and 11B illustrate a lens formed according to the present disclosure, along with its theoretical side view;

FIGS. 12A and 12B illustrate a lens formed according to the present disclosure, along with its theoretical side view;

FIG. 13 is a flowchart of a method for forming a lens in accordance with an illustrative embodiment;

FIG. 14 is a flowchart of a method for performing lens calculations to form a customized lens according to an illustrative embodiment;

FIG. 15 is a block diagram of a system for controlling a lens fabrication process according to an illustrative embodiment;

FIG. 16 is a graph depicting the relationship between energy needed and UV absorber concentration for lens formation;

FIG. 17A is a graph depicting the relationship between a refractive index of a plastic material and pulse energy of femtosecond laser pulses; and

FIG. 17B is a graph depicting the relationship between a refractive index of a plastic material and number of femtosecond laser pulses.

### DETAILED DESCRIPTION

A common problem with the molding technique for IOL formation is that it is a very expensive way to make a customized lens. For most patients, only an approximate approach to clear vision is obtained, and for some patients, the optimal diopter power can be wrong by 0.25 or more. Moreover, such lenses generally are not as effective for patients who have an abnormally shaped cornea, including some who have undergone a cornea procedure, such as in Laser-Assisted In Situ Keratomileusis (LASIK) surgery.

A common problem with the lathing and milling technique of IOL formation is that the lens is machined at low temperatures for ease of manufacture, but the optical properties of the lens material at low temperatures can differ fairly significantly from the optical properties of the lens at body temperature. Further, warming of the lens formed by lathing and milling results in absorption of moisture, which can change the dimensions of the lens and affect its corresponding diopter power.

Furthermore, some patients have lenses possessing natural corneal spherical aberrations or corneal astigmatisms, or lenses that have been artificially altered via surgical procedures such as LASIK. Commercially available IOLs formed from one of the two conventional manufacturing techniques generally cannot correct these optical defects because manufacturers of IOLs would be required to inventory numerous different types of IOLs, all varying in dioptic power and aspheric and toric features.

Novel aspects of the present disclosure recognize the need to create a custom IOL to adjust diopter and/or modify properties such as toricity, asphericity, and multifocality without cutting or ablating the lens. The custom IOL can be formed by introduction of energy by a femtosecond laser sufficient to modify UV absorbers dispersed throughout the IOL to alter the refractive properties of the IOL. In some embodiments, the energy can cause a chemical reaction within the IOL causing the formation of compounds of the UV absorber. In these embodiments or others, the energy can break one or more bonds within the UV absorber to form new or modified compound, e.g., new or modified UV absorbers or other compounds within the IOL, altering the refractive capabilities of the IOL. Modification of the UV absorbers can also induce changes in the polymeric lens material of the IOL to control the path of light through the IOL. For example, the femtosecond laser can be used to create structures within the polymeric lens material of the IOL to direct light towards or away from a target location, like a retina. Thus, the resultant IOL can have one or more structures that that change a refractive index that can improve a patient's vision, and one or more additional structures that can redirect light through the IOL.

A typical application for of the novel aspects of this disclosure can include correcting the post-operational residual refractive error of an intraocular lens that has already been implanted in a patient's eye.

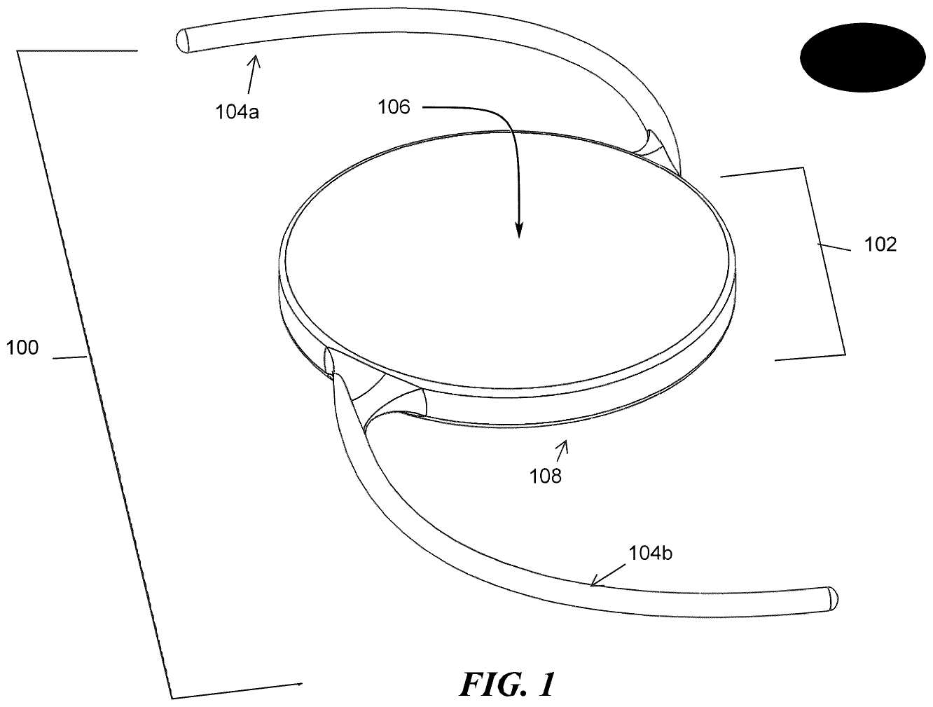

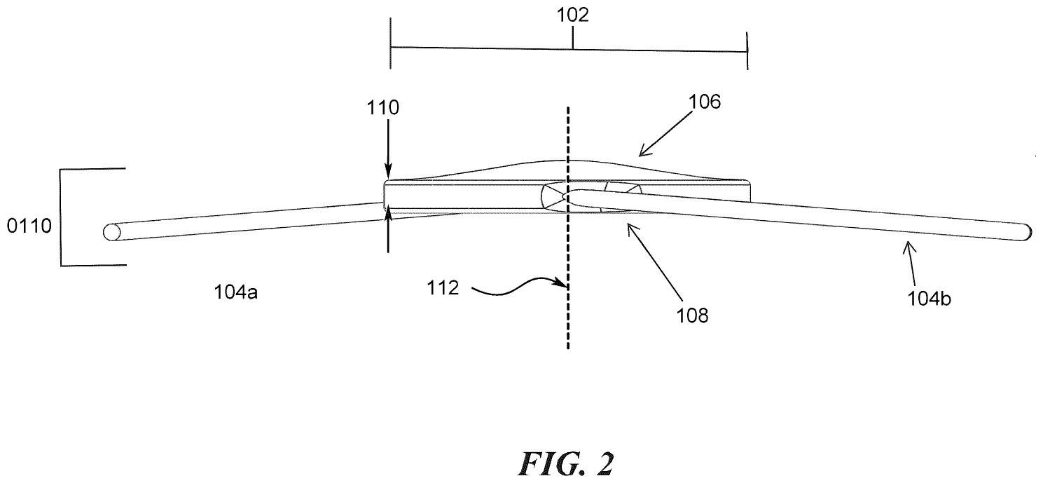

FIGS. 1 and 2 are various views of an IOL. In particular, FIG. 1 is a perspective view of IOL 100, and FIG. 2 is a side view of IOL 100. IOL 100 is formed from a central body 102 from which a pair of simplified haptics 104a and 104b (collectively “haptics 104”) extend. The central body 102 has an anterior surface 106 separated from a posterior surface 108 by a thickness 110. The terms “anterior” and “posterior” refer to surfaces of a lens as it is normally placed in the human eye, with the anterior surface 106 facing outwardly and the posterior surface 108 facing inwardly toward the retina. From FIG. 2, it can be seen that the IOL 100 has an optical axis 112 passing perpendicularly through the center of the central body 102, defining the path along which light propagates through the IOL 100. In at least one embodiment, the optical axis 112 coincides with the mechanical axis of the lens.

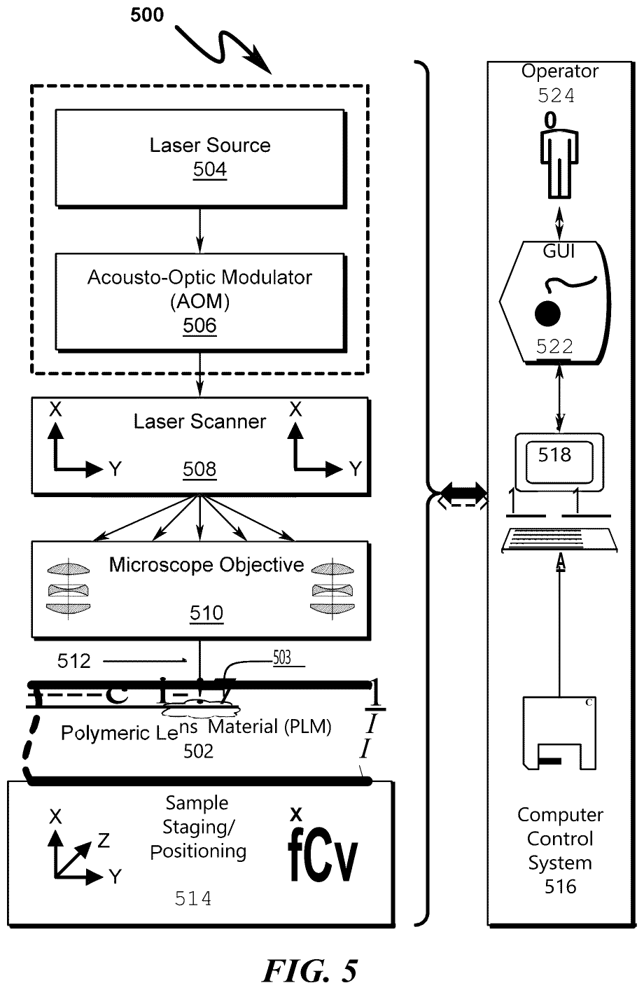

The present disclosure provides a system, e.g., system 500 shown and described in FIG. 5, and a corresponding method for shaping the anterior surface 106 and posterior surface 108, and also altering the refractive capabilities of the IOL 100. The refractive capabilities can be altered by a laser with operating parameters selected to split the bonds of the UV absorbers dispersed throughout the IOL 100 for changing its refractive properties, but without causing undesirable cross-linking or mechanical damage to the IOL 100. The resultant IOL 100 can be formed in a spherically symmetric fashion, or in some embodiments, asymmetrically or in a custom pattern that corresponds to specific topical irregularities of the cornea of an individual patient.

UV absorbers are molecules that absorb UV radiation. UV absorbers can be dispersed throughout the polymeric material blank from which the IOL 100 is formed. Non-limiting examples of UV absorbers that can be incorporated into IOL 100 can include derivatives of benzotriozoles, such as 2-(5-chloro-2-H-benzotriazol-2-yl)-6-(1,1-dimethyl-ethyl)-4-(propyenyloxy-propyl)phenol, and benzophenol derivatives, such as 3-vinyl-4-phenylazophenylamine, which is a yellow dye that absorbs at a wavelength of 390 nm. Other examples can include 2-Propenoic acid, 2-methyl-,2-[(4,6-diphenyl-1,3,5-triazin-2-yl)-3-hydroxyphenoxy]ethyl ester; 2-(4-Benzoyl-3-hydroxyphenoxy)ethyl acrylate (cyasorb UV-2098); 4-(2-acryloxyethoxy)-2-hydroxybenzophenone; 2-[3-(2H-Benzotriazol-2-yl)-4-hydroxyphenyl]ethyl methacrylate (BZL); 4-[(E)-Phenyldiazenyl]phenyl-methacrylate; and 2-(5-Chloro-2H-benzotriazole-2-yl)-6-(1,1-dimethyl-ethyl)-4-methyl-phenol (Tinuvin 326). The amount of UV absorber included in the polymeric blank material can be at least 0.01 wt %, and in some embodiments, the amount of UV absorber included in the polymeric blank material can be between 0.01-1 wt % of the IOL 100.

UV absorbers exposed to a laser, i.e., a particular form of UV radiation, can alter a molecular structure of the UV absorbers to create new or modified UV absorbers providing an IOL with different refractive characteristics. For example, a native UV absorber dispersed throughout a polymeric blank material from which the IOL 100 is formed, can be cleaved into two separate UV absorbers. In addition, or in the alternative, the native UV absorber can be modified to form a different UV absorber. In either case, the laser can create new or modified UV absorbers by breaking one or more bonds of the native UV absorber. The bonds can be polar bonds or non-polar bonds, and/or single, double, or triple bonds.

As an example, the following is a chemical structure of a high molecular weight, low volatility benzotriazole UV absorber:

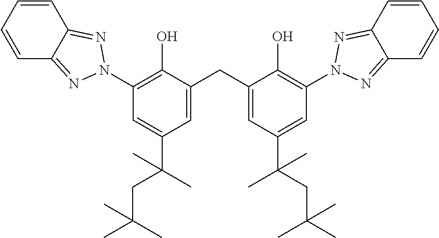 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

The laser can cleave the UV absorber into two separate benzotriazole UV absorbers. In a first embodiment, the UV absorber can be split into two identical UV absorbers of lower molecular weight, as shown below:

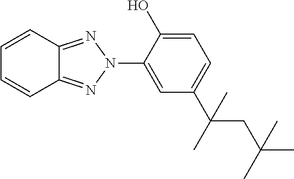 [SMILES: CC(C)(C)CC(C)(C)c1ccc(O)c(-n2nc3ccccc3n2)c1]

In a second embodiment, the UV absorber can be split into slightly two different UV absorbers, e.g., one of which is shown above and the other of which includes an extra methyl group adjacent to the hydroxyl group.

In another example, the laser can remove functional groups from the native UV absorber to create a different UV absorber that imparts a different refractive capability to the IOL. The native UV absorber can be a general purpose benzotriazole UV absorber as shown below:

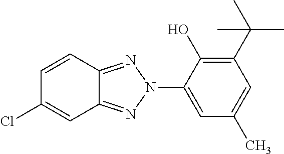 [SMILES: Cc1cc(-n2nc3ccc(Cl)cc3n2)c(O)c(C(C)(C)C)c1]

The laser can remove functional groups from the UV absorber to form a modified UV absorber. For example, the laser can remove the chlorine and butyl functional group to form the following UV absorber:

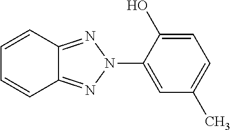 [SMILES: Cc1ccc(O)c(-n2nc3ccccc3n2)c1]

In some instances, the UV absorber can be affected to form a new or different compound which could nevertheless change the refractive capability of the IOL.

Exposure of the polymeric blank material to a laser for extended periods of time accelerates free electrons in the polymeric blank material to high energies that can cause collisions with other atoms or molecules, forming secondary free carriers. The increasing density of free carriers can eventually result in an optical breakdown that forms an electrically conductive plasma that can lead to mechanical damage. However, a strongly focused femtosecond laser with short pulse duration can produce small, precise localized defects without cracks before optical breakdown is achieved. The UV absorbers dispersed throughout the polymeric blank material convert the UV radiation of the laser into heat, which can be distributed to the polymeric blank material. Without wishing to be bound by theory, it is hypothesized that the presence of breakdown of UV absorbers, either by forming a modified UV absorber or by splitting the UV absorber into two or more lower molecular weight UV absorbers, or by splitting the UV absorber into a new or different compound that is no longer considered a UV absorber, is an endothermic process that results in structural formations within the polymeric blank material that differs from the structural formations formed in the polymeric blank material which occur when UV absorbers are unaltered, i.e., when the UV absorbers are unaltered, the polymeric blank material is exposed to relatively higher heat.

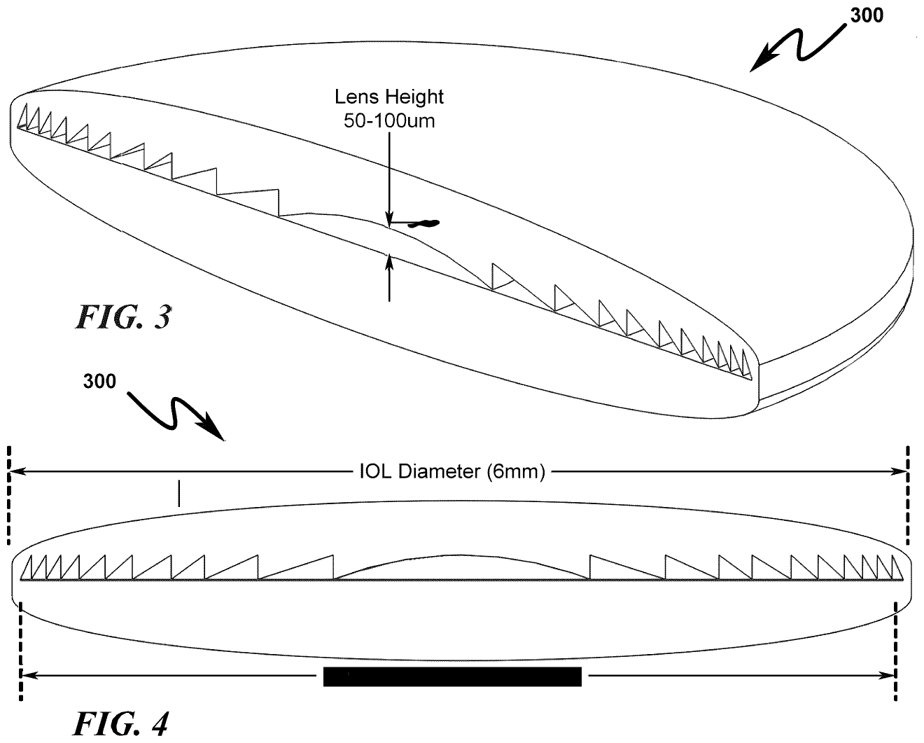

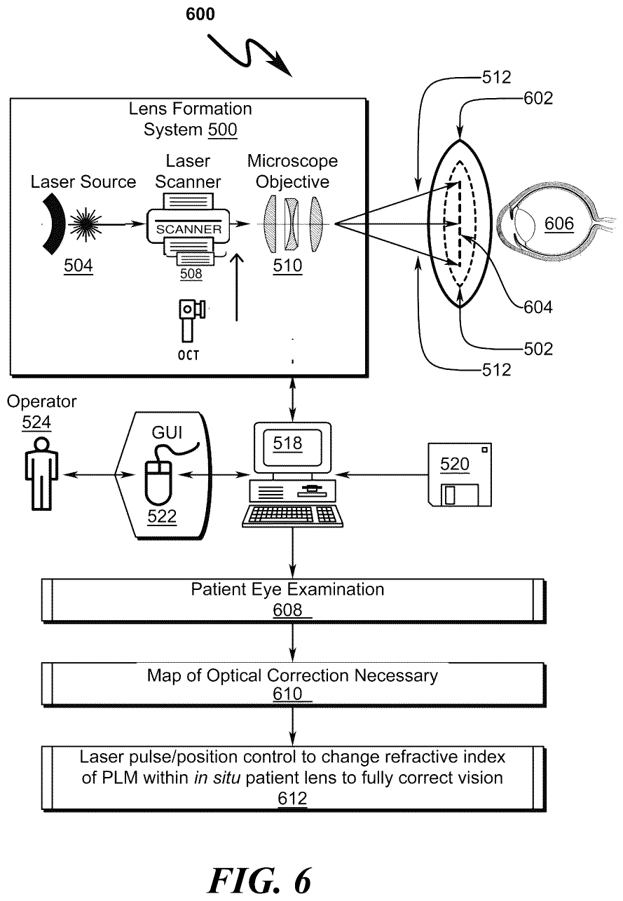

FIGS. 3 and 4 are views of a lens that can be formed by the application of laser radiation as described in this disclosure. In particular, FIG. 3 is a sectioned perspective view of an IOL 300, and FIG. 4 is a side view of the sectioned IOL 300 depicted in FIG. 3. The IOL 300 can be modified to include lens structure 302 formed within the IOL 300. In this illustrative embodiment, the lens structure 302 is a refractive index shaping (RIS) lens. The RIS lens can be formed by a femtosecond laser focused within the body of the IOL 300 as described in more detail in FIGS. 5 and 6 that follow. In a non-limiting example, the femtosecond laser is configured with a wavelength between 500-530 nm, or more particularly between 510-120 nm. In a particular embodiment, the femtosecond laser is configured to create the lens structure 302 within the IOL 300 with a wavelength that is about 515 nm. In another embodiment, the femtosecond laser is configured to form the lens structure 302 with a wavelength that is twice the wavelength of the sculpting laser beam used to sculpt a polymeric lens material into an IOL. For example, if the sculpting laser beam is 1040 nm, then the lens structure 302 is formed with a femtosecond laser having a wavelength of about 520 nm.

FIG. 5 is a block diagram of a system for creating an IOL according to an illustrative embodiment. The system 500 is generally configured to irradiate a polymeric lens material 502 with laser radiation from laser source 504 to produce a change in the UV absorbers disposed in a selected region 503 within the polymeric lens material 502. The change in the UV absorbers results in a change of refractive properties of the polymeric lens material 502 and in the resultant IOL formed from the polymeric lens material 502. For example, the change in the UV absorbers can be new or modified UV absorbers, as previously discussed.

The laser source 504 can be configured to generate pulsed laser radiation that can be controlled by an acousto-optic modulator 506 to produce a predetermined laser pulse train having specified energy and pulse timing characteristics. In some embodiments the laser source 504 and the acousto-optic modulator 506 may be integrated together, e.g., into a single laser source module. The pulsed laser radiation generated by the laser source 504 and the acousto-optic modulator 506 can be transmitted to a laser scanner 508 that is configured to distribute the laser pulses in an X-Y plane across an input area of a microscope objective 510. The microscope objective 510 incorporates a numerical aperture configured to accept the distributed, pulsed laser radiation and produce a focused laser radiation output 512. This focused laser radiation output 512 is then transmitted by the microscope objective 510 to the selected region 503 of the polymeric lens material 502. The position of the selected region 503 in the polymeric lens material 502 exposed to the focused laser pulses 512 can be controlled by the laser scanner 508 and a sample staging system 514 that mechanically positions the polymeric lens material 502 to allow the focused laser pulses 512 to be properly localized at the selected region 503 of the polymeric lens material 502.

The system 500 may optimally operate under control of a computer control system or computing device 516 incorporating a computer 518 executing software read from a computer readable medium 520 and providing a graphical user interface (GUI) 522 from which an operator or user 524 may direct the overall operation of the system 500 for controlling the modification of UV absorbers within the polymeric lens material 502.

FIG. 6 is a block diagram of an overall system 600 that includes system 500 for creating a lens structure 602 according to an illustrative embodiment. As previously mentioned, the system 500 generates focused laser pulses 512 directed to one or more selected regions of the lens structure 602, all or some of which is formed of polymeric lens material 502, to irradiate the selected region in a two or three-dimensional pattern 604. The focused laser pulses 512 modifies UV absorbers present in the polymeric lens material 502 to cause changes in the refractive index of the polymeric lens material 502.

The lens structure 602 can be implanted within a human eye 606 and the polymeric lens material 502 modified in situ. The lens formation system 500 is typically operated under control of a computer 518, as previously described. With respect to the above-mentioned in situ lens formation application, the computer readable medium 520 may incorporate software implementing methods to perform an automated patient eye examination 608 to determine the non-idealities in the patient's vision, from which a map of optical corrections 610 necessary to improve the patient's vision is generated, followed by automated laser pulse/position control procedures to change in situ the refractive index of PLM within the patient lens to correct the patient vision 612.

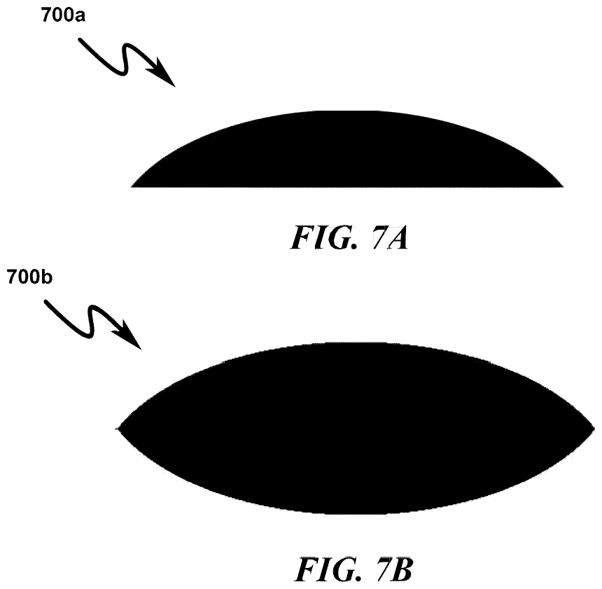

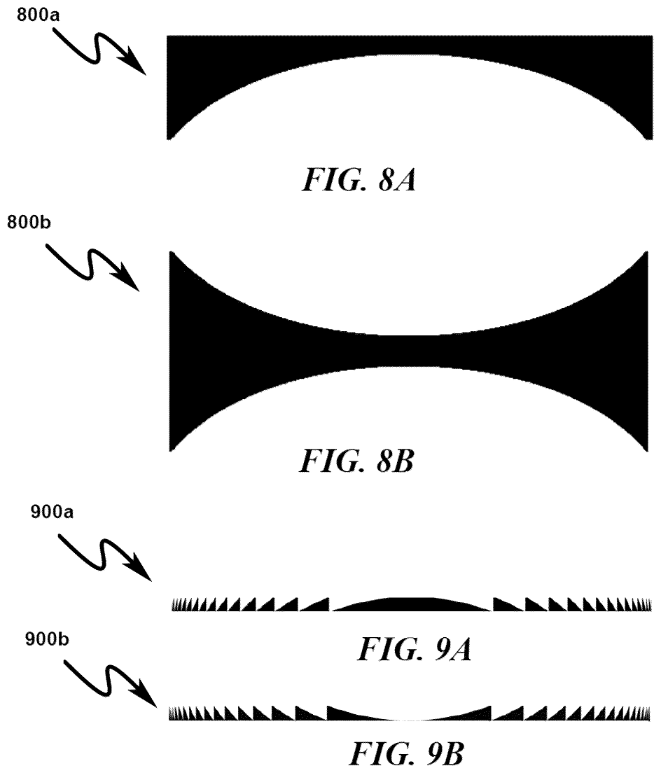

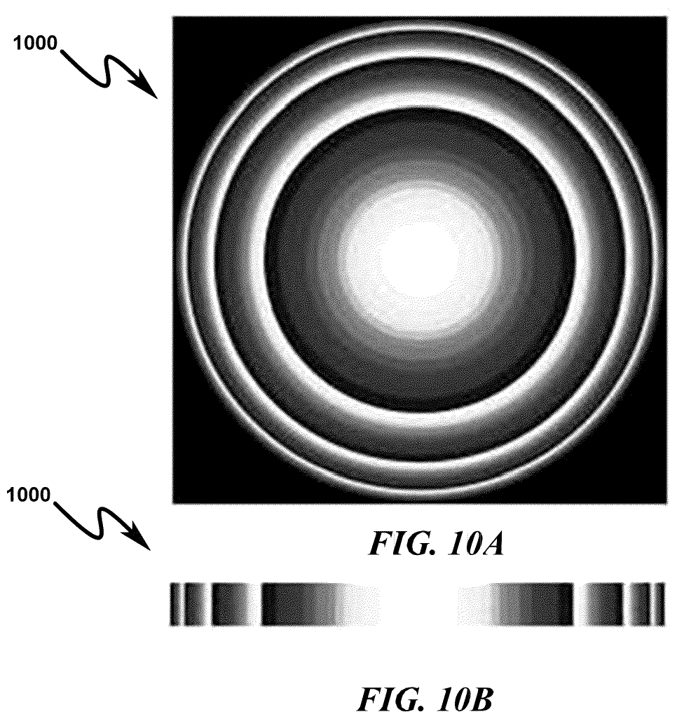

FIGS. 7-10 are illustrations of various lens structures that can be formed by system 600. In particular, FIGS. 7A and 7B depict a convex lens 700a and a biconvex lens 700b, respectively; FIGS. 8A and 8B depict a concave lens 800a and a biconcave lens 800b, respectively; FIGS. 9A and 9B depict a phase wrapping convex lens 900a and a phase wrapping concave lens 900b, respectively; and FIGS. 10A and 10B depict a plan view and a side view of a refractive index gradient lens 1000, respectively. The grayscale values are used to represent the energy per pulse; therefore, 256 variations of the power between 0% and 100% are possible, which allows for the precise shaping of a single layered lens. The plan view of a refractive index lens 1000 shown in FIG. 10A shows the different zones of an original convex phase wrapping lens. Each original discussed lens type data information can be compressed to one single layer. The side view of the refractive index gradient lens 1000 depicted in FIG. 10B 1502 shows the energy distribution at each spot for one horizontal slice through the center of the lens 1000.

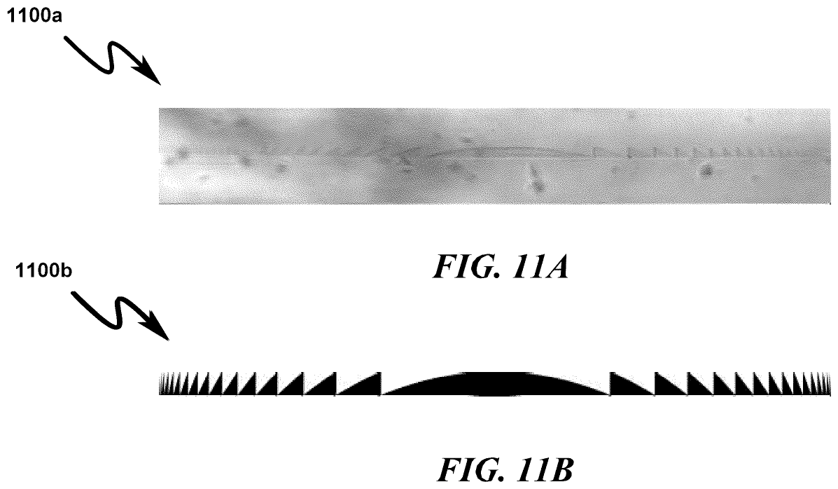

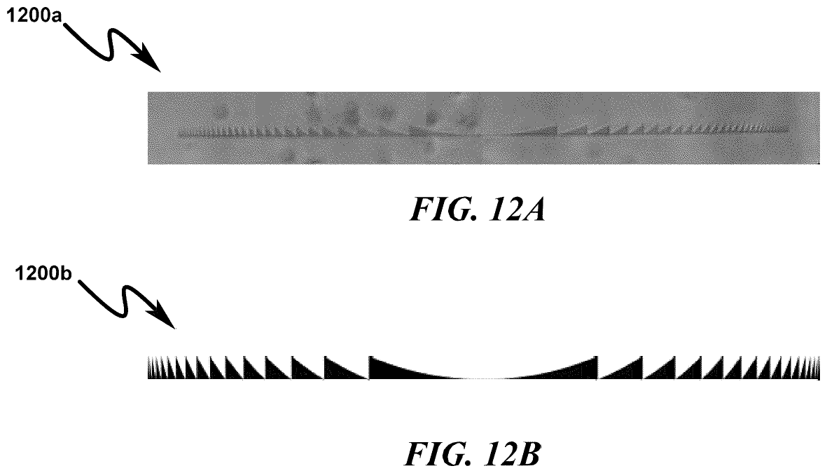

FIGS. 11A and 11B illustrate a lens structure formed according to the present disclosure, along with its theoretical side view. In particular, FIG. 11A depicts a phase wrapping convex lens structure 1100a, and FIG. 11B depicts a theoretical side view 1100b of the phase wrapping convex lens structure 1100a. The phrase wrapping convex lens structure 1100a provides a negative refractive index change, i.e. a negative diopter reading. FIGS. 12A and 12B illustrate another lens structure formed according to the present disclosure, along with its theoretical side view. Specifically, FIG. 12A depicts a phase wrapping concave lens structure 1200a, and FIG. 12B depicts a theoretical side view 1200b of the phase wrapping concave lens structure 1200a. The phase wrapping concave lens structure 1200a provides a positive refractive index change, i.e., a positive diopter reading. The refractive index changes in the lens structures 1100a and 1100b are induced by the UV absorber change inside the polymeric lens material of the lens structure.

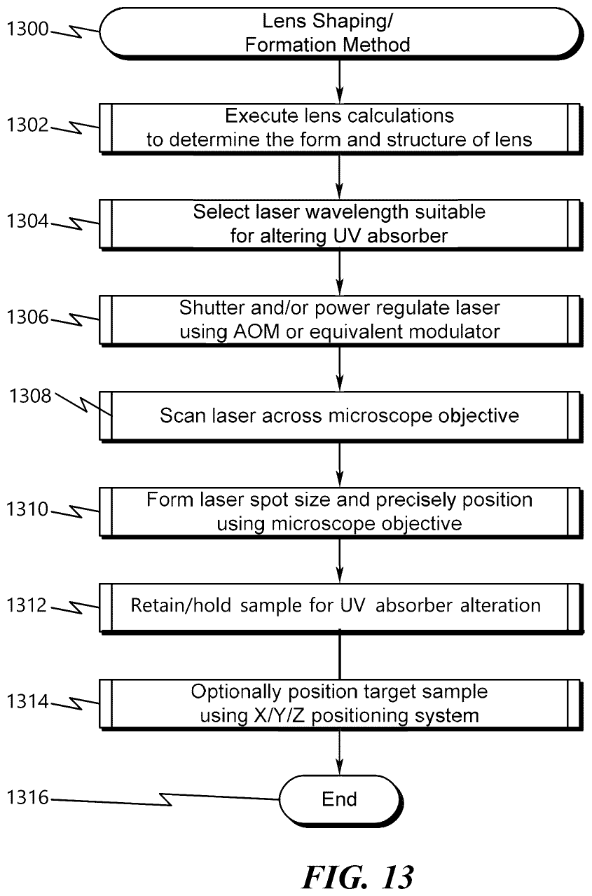

FIG. 13 is a flowchart of a method for forming a lens in accordance with an illustrative embodiment. Method 1300 can be used to form a lens structure of arbitrary complexity within a polymeric lens material. In step 1302, lens calculations are performed to determine the form and structure of the lens structure. The lens calculations can be used to determine the lens diopter and curvature as a function of the selected polymeric lens material. In step 1304, a suitable wavelength is selected for inducing the desired UV absorber alteration in the polymeric lens material. In step 1306, modulated laser pulses are generated to precisely shape a lens structure within the polymeric lens material. In a non-limiting embodiment, the laser pulses are modulated with an acousto-optic modulator, as previously discussed, which can serve as a shutter and variable power attenuator. The acousto-optic modulator can be controlled by the input images of the calculated lens information step providing the laser power information for the irradiated area. In step 1308 the laser pulses are scanned across a microscope objective. The scanner distributes the power to the correct location and the microscope objective focuses the pulsed laser beam to the desired focus spot inside the polymeric lens material in step 1310. In step 1312, the polymeric lens material is retained in a sample holder to allow for the UV aborbers to undergo alteration. The polymeric lens material sample can be optionally positioned using a X-Y-Z positioning system to allow the shaping of the polymeric lens material sample to a lens structure. In some embodiments, the stage system could also be replaced with a mirrored laser arm which ends with the microscope objective. The mirrored arm in this case would not only replace the stage system but the whole camera and scanner board.

This method can come to an end 1316, or in some examples there may be a return to complete the same or similar operations in the polymeric lens material. This general method may be modified depending on a number of factors, with rearrangement and/or addition/deletion of steps anticipated by the scope of the present disclosure. Integration of this and other preferred exemplary embodiment methods in conjunction with a variety of preferred exemplary embodiment systems described herein is anticipated. Method 1300 may be applied to one or more layers within a polymeric lens material to achieve formed lens structures of arbitrary complexity.

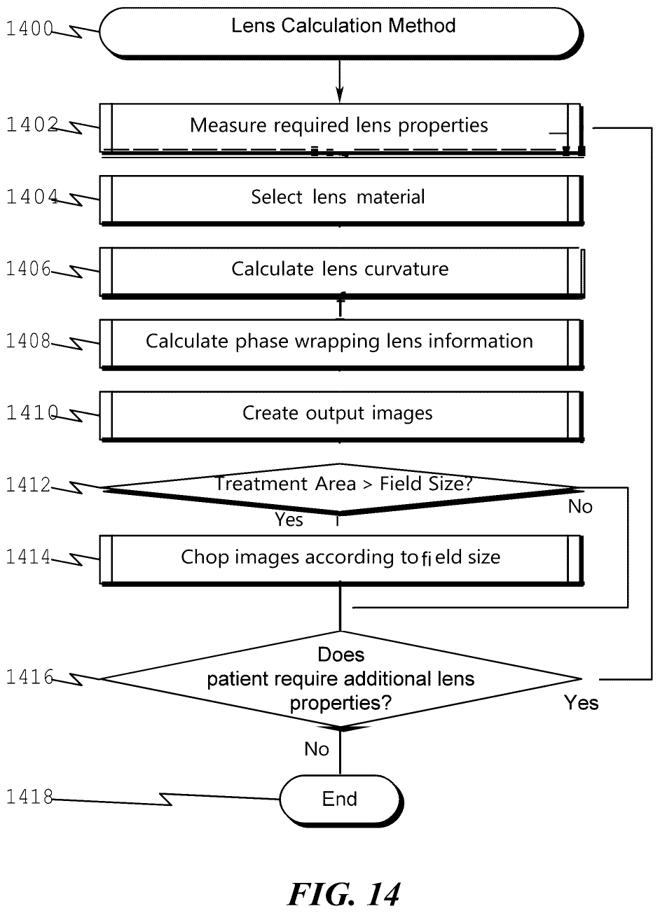

FIG. 14 is a flowchart of a method for performing lens calculations to form a customized lens according to an illustrative embodiment. The steps of method 1400 can be implemented in step 1302 from method 1300.

Method 1400 begins at step 1402 by measuring and/or determining required lens properties for desired optical performance. These properties can be obtained by an examination of a patient and measuring any existing aberrations and determining any needed diopter changes (Dpt).

In step 1404, a lens material is selected. The lens material has a refractive index n.

In step 1406, a lens curvature is calculated. The lens curvature can be calculated by the following equation:

$$C = \frac{Dpt}{( n ′ - n )} \qquad ( 1 )$$

where Dpt is the needed diopter changes, n is the refractive index of the lens material, and n′ is the desired refractive index after lens shaping.

The lens curvature can also be calculated according to the following equation:

$$C = \frac{1}{r}$$

(2)
where r is the lens radius.

The lens radius, r, can be calculated by the following equation:

$$r = \frac{{h}_{Lens}^{2} + {w}_{Lens}^{2}}{2 {h}_{Lens}} \qquad ( 3 )$$

where wLens is the lens diameter and hLens is the lens depth.

In step 1406 phase wrapping lens information is calculated. In step 1408, output images that correspond to the desired phase wrapping lens characteristics are created.

In step 1410, a determination is made as to whether the lens treatment area is larger than the objective field size. If the lens treatment area is larger than the objective field size, then method 1400 proceeds to step 1414 and the output images are chopped into segments that fit within the field size. The method 1400 then proceeds to step 1416 where a determination is made as to whether additional lens properties are required. If additional lens properties are not required, then method 1400 terminates at step 1418. If additional lens properties are required, then the method 1400 returns to step 1402.

Returning to step 1412, if the determination is made that the lens treatment area is not larger than the objective field size, then method 1400 proceeds to step 1416.

This method 1400 may be modified depending on a number of factors, with rearrangement and/or addition/deletion of steps anticipated.

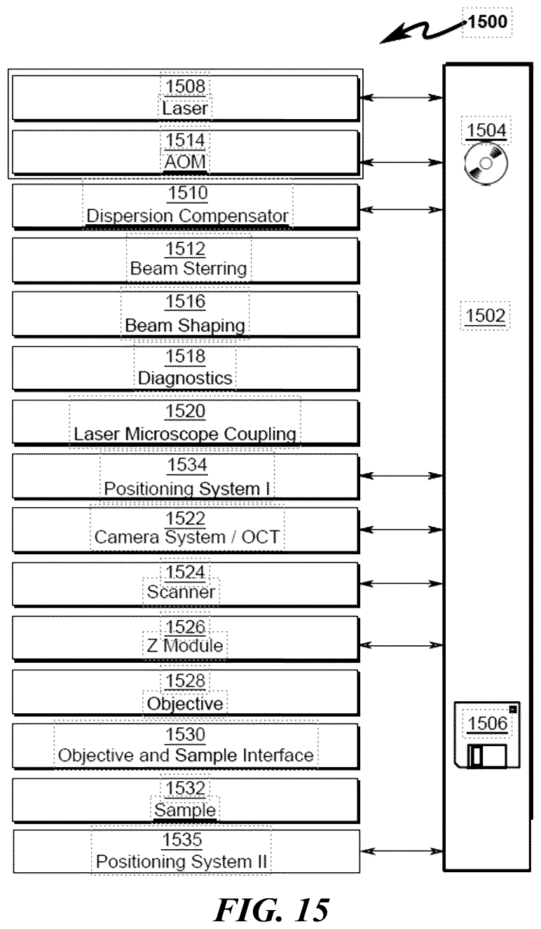

FIG. 15 is a block diagram of a system 1500 for controlling a lens fabrication process according to an illustrative embodiment. A computer system 1502 operating under control of software read from a computer readable media 1504, 1506 is used to control and supervise the overall lens fabrication process.

A non-limiting embodiment of the system 1500 can include a laser 1508 with a wavelength suitable to treat the sample 1532, i.e., the desired polymeric lens material, and an energy-per-pulse sufficient to alter UV absorbers disposed therein to change the refractive index of the selected area of the sample 1532. The laser radiation from the laser 1508 can be pre-compensated by a dispersion compensator 1510, which allows a pulse width around less than 400 fs. Without the feature the pulse width at the target would be larger because the pulse width gets longer when passing through an optical media like a lens. A longer pulse with more heat would occur on the treatment area, making the process a little more imprecise and the treatment time a little longer. This feature therefore is optional.

The beam steering unit 1512 can be used to modify the diameter of the laser radiation output to fit the specifications of the acousto-optic modulator 1514, which is used to modulate the number of pulses and the energy per pulse which will be directed to the treatment area.

After the beam has passed through the acousto-optic modulator 1514, additional beam shaping may be required to fit the system. For example, the beam of laser radiation may have a diameter that needs to be enlarged to fit the objective 1528, to allow the use of the numerical aperture of the objective 1528. Beam shaping 1516 can shape the beam of laser radiation to fit the objective 1528.

A diagnostics system 1518 can be used to measure the wavelength, energy per pulse, and the pulse width of the laser beam, which allows for the safe and repeatable use of the system 1500. The diagnostics system 1518 can power down the system 1500 upon detecting abnormal operational variables.

Laser microscope coupling device 1520 can be used to redirect the laser beam into the laser microscope head. Depending on the system setup and laser orientation the laser microscope coupling device 1520 can contain one or more mirrors to redirect the laser beam to the needed position.

The camera system 1522 can be used to position the sample 1532 towards the microscope objective 1528. It also is used to find the correct Z-location, depending on the curvature of sample 1532. Additionally, the camera system 1522 can be used for tracking purposes.

The scanner 1524 can be used to distribute the laser spot on the XY plane. Different scanners can be used for this purpose. In some embodiments, the scanner 1524 can be controlled by the same software used to control the acousto-optic modulator 1514 so the laser spot can be positioned on the XY plane and the acousto-optic modulator 1514 can control the energy per pulse for that spot.

The Z Module 1526 can be used to allow an extra focusing element in the system 1500, which can be used for tracking purposes for a plane in a different Z location than the shaping plane. The Z module 1526 can also be used to change the Z location of the sample 1532 during the shaping process.

The objective 1528 focuses the beam on the sample 1532 and determines the spot size. With a larger spot size, a larger energy per pulse is required, it therefore has to be fitted to the laser source and the required precision of the process. Additionally, the objective 1528 provides the field size of the shaping process. If the field size of the objective 1528 is smaller than the required lens, additional hardware may be needed for the lens shaping.

The objective and sample interface 1530 is an application-dependent medium that is applied in the space between the sample 1532 and the objective 1528 to reduce scattering and improve heat dissipation. The objective and sample interface 1530 can be water or a gel.

The sample 1532 can comprise different optical mediums and could for example be a polymer lens material.

The positioning system 1535 can be used to position the blocks comprising the objective field sizes relative to each other to allow the shaping of a larger structure. It can also be used to move the sample 1532 in the Z direction.

One skilled in the art will recognize that a particular invention embodiment may include any combination of the above components and may in some circumstances omit one or more of the above components in the overall system implementation.

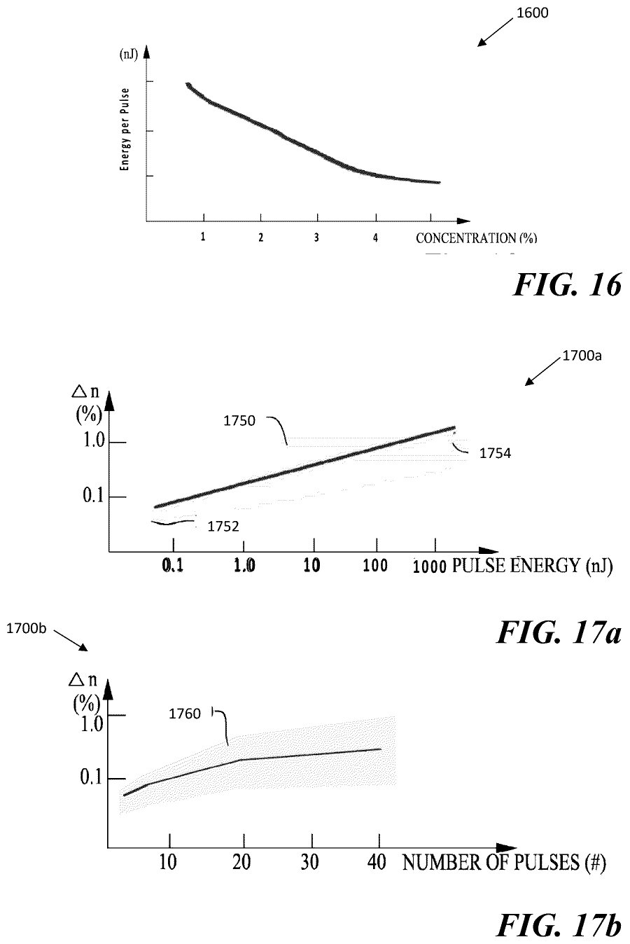

FIG. 16 is a graph depicting the relationship between energy and UV absorber concentration for lens formation. In particular, the graph 1600 depicts the threshold energy (I) (nJ) necessary for achieving permanent structural change in plastic material as a function of concentration (%) of an aromatic UV absorber. The typical characteristic demonstrates a strong dependence of the threshold energy on the concentration of the UV absorber, indicating the enhancement of the local permanent structural change with the concentration of the UV absorber. It is desirable to have as little energy as possible used for this purpose, because exposure to excess energy can result in a cross-linking or other undesirable mechanical changes in the lens or lens body.

The local interaction of the molecules of the plastic host results in a localized, partial increase Δn of the refractive index n. At a concentration of 0.8% of the UV absorber, as used in commercial intraocular lens materials, a threshold energy of about 0.1 nJ is required.

These changes can be targeted on particular sections of the polymeric lens material that include the original or first UV absorber, which when exposed to a laser source can then be modified into new or multiple UV absorbers that were not previously present. Similarly, multiple passes with a laser source may cause multiple changes in the UV absorber structure, and thereby allowing for different lens structures, combinations, phases, gradients, or other objectives to be met.

FIG. 17a is a graph 1700a depicting the relationship between a refractive index of a plastic material and pulse energy of femtosecond laser pulses. In particular, FIG. 17a depicts the change Δn of the refractive index as a function of the pulse energy (nJ). The curve 1750 in FIG. 17a demonstrates that an increasing pulse energy from 0.1 nJ to 8 nJ enhances the change Δn of the refractive index n from approximately 0.1% to approximately 1.0%. The threshold for the initial occurrence of a measurable change Δn of the refractive index n is denoted at position 1752 of the curve 1750. At a pulse energy level of approximately 8 nJ, the threshold for photo disruption of the plastic material is reached, resulting in collateral damage of the material and opacifications, facilitating undesirable scattering losses of the light that is transmitted through the plastic material. As can be seen from curve 1750, the range of possible pulse laser energies extends over two orders of magnitude, from 0.05 nJ to 8 nJ, allowing for a safe operation of the manufacturing process which occurs at the lower end of the range, at a pulse energy.

FIG. 17b is a graph 1700b depicting the relationship between a refractive index of a plastic material and number of femtosecond laser pulses. In particular, FIG. 17b depicts the change Δn of the refractive index as a function of the number of pulses in the focal volume at a fixed pulse energy (e.g. 0.2 nJ). The curve 1760 indicates that the cumulative effect of approximately 50 laser pulses in the focal volume yields refractive index changes Δn of the order 1%, sufficient for achieving an optical path length difference (OPD=Δn×thickness) of 1.0 waves in a plastic material layer of 50 μm thickness, choosing a low pulse energy of 0.2πJ.

The present disclosure may include a computing device that can include any of an application specific integrated circuit (ASIC), a microprocessor, a microcontroller, a digital signal processor (DSP), a field-programmable gate array (FPGA), or equivalent discrete or integrated logic circuitry. In some examples, the system may include multiple components, such as any combination of one or more microprocessors, one or more microcontrollers, one or more DSPs, one or more ASICs, or one or more FPGAs. It would also be understood that multiples of the circuits, processors, or controllers could be used in combination or in tandem, or multithreading. Additionally, it would be understood that a browser or program could be implemented on a mobile device or mobile computing device, such as, a phone, a mobile phone, a cell phone, a tablet, a laptop, a mobile computer, a personal digital assistant (PDA), a processor, a microprocessor, a micro controller, or other devices or electronic systems capable of connecting to a user interface and/or display system. A mobile computing device or mobile device may also operate on or in the same manner as the computing device disclosed herein or be based on improvements thereof.

The components of the present disclosure may include any discrete and/or integrated electronic circuit components that implement analog and/or digital circuits capable of producing the functions attributed to the modules herein. For example, the components may include analog circuits, e.g., amplification circuits, filtering circuits, and/or other signal conditioning circuits. The components may also include digital circuits, e.g., combinational or sequential logic circuits, memory devices, etc. Furthermore, the modules may comprise memory that may include computer-readable instructions that, when executed cause the modules to perform various functions attributed to the modules herein.

Memory may include any volatile, non-volatile, magnetic, or electrical media, such as a random-access memory (RAM), dynamic random-access memory (DRAM), static random-access memory (SRAM), read-only memory (ROM), non-volatile RAM (NVRAM), electrically-erasable programmable ROM (EEPROM), flash memory, hard disks, or any other digital media. Additionally, there may also be a tangible non-transitory computer readable medium that contains machine instructions, such as, a (portable or internally installed) hard drive disc, a flash drive, a compact disc, a DVD, a zip drive, a floppy disc, optical medium, magnetic medium, or any other number of possible drives or discs, that are executed by the internal logic of a computing device. It would be understood that the tangible non-transitory computer readable medium could also be considered a form of memory or storage media.

While this disclosure has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Brief Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

We claim:

1. A system for forming a refractive lens, the system comprising:
a pulsed laser configured to produce a pulsed laser output; and
an optical device configured to form a focused laser output from the pulsed laser output and direct the focused laser output at a polymeric lens material containing a UV absorber compound, wherein the focused laser output is configured to alter a molecular structure of the UV absorber compound to form a new or modified compound that imparts a different refractive property in the refractive lens.

2. The system of claim 1, wherein the optical device comprises a focusing device and a scanner.

3. The system of claim 2, wherein:
the scanner is configured to distribute the pulsed laser output in a plane to form a distributed laser output, and
the focusing device is configured to focus the distributed laser output to produce a focused laser output.

4. The system of claim 2, wherein the focusing device is a microscope objective.

5. The system of claim 3, wherein the microscope objective includes a numerical aperture.

6. The system of claim 1, wherein the molecular structure altered by the focused laser output includes one or more polar bonds.

7. The system of claim 1, wherein the molecular structure altered by the focused laser output includes one or more non-polar bonds.

8. The system of claim 1, wherein the molecular structure altered by the focused laser output includes one or more single bonds.

9. The system of claim 1, wherein the molecular structure altered by the focused laser output includes one or more double bonds.

10. The system of claim 1, wherein the molecular structure altered by the focused laser output includes one or more triple bonds.

11. The system of claim 1, wherein the change of the molecular structure results in a positive refractive index change.

12. The system of claim 1, wherein the change of the molecular structure results in a negative refractive index change.

13. The system of claim 1, wherein at least one UV absorber compound is converted into two new UV absorber compounds.

14. The system of claim 1, wherein at least one UV absorber compound is converted into a new UV absorber compound.

15. The system of claim 1, wherein at least one UV absorber compound comprises at least two UV absorber compounds that are converted into the new UV absorber compound and a second new absorber compound.

16. The system of claim 1, wherein the pulsed laser is a femtosecond laser.

17. The system of claim 1, wherein the different refractive property is a positive refractive index change.

18. The system of claim 1, wherein the different refractive property is a negative refractive index change.

19. The system of claim 1, wherein the formation of the new or modified compound is based on a power level or a power intensity of the pulsed laser.

20. The system of claim 1, wherein the pulsed laser output is further configured to alter a molecular structure of the polymeric lens material along with the molecular structure of the UV absorber compound.

21. The system of claim 1, wherein the different refractive property is a direction of light relative to a target location.

22. The system of claim 1, wherein the target location is a retina and the direction of light is either towards the retina or away from the retina.

23. A system for altering an existing refractive lens, the system comprising:
a pulsed laser configured to produce a pulsed laser output; and
an optical device configured to form a focused laser output from the pulsed laser output and direct the focused laser output at a polymeric lens material containing a UV absorber compound, wherein the focused laser output is configured to alter a molecular structure of the UV absorber compound to form a new or modified compound that transforms a portion of the refractive lens such that light hitting that portion of the refractive lens is directed to or away from away from the retina.
